(19) United States
(12) Patent Application Publication
(10) Pub. No.: US 2003/0130595 A1
(43) Pub. Date: Jul. 10, 2003

(54) Health improvement systems and methods

(76) Inventor: James R. Mault, Evergreen, CO (US)

Correspondence Address: Allen M. Krass, Gifford, Krass, Groh, Sprinkle,, Anderson & Citkowski, P.C., 280 N. Old Woodward Ave. Suite 400, Birmingham, MI, 48009-5394, US

(21) Appl. No.: 10/339,262

(22) Filed: Jan. 10, 2003

Related U.S. Application Data
(63) Continuation-in-part of application No. PCT/US01/41689, filed on Aug. 13, 2001.

(30) Foreign Application Priority Data
Jan. 8, 2003 (US) 10/338,391

(51) Int. Cl. A61B 10/00

(52) U.S. Cl. 600/567

(57) ABSTRACT

A system for assisting a person achieve a health related goal comprises a local computer, adapted to receive a physiological parameter or other health related data relating to a subject, and a remote computer, in communication with the local computer over a communications network, adapted to receive data from the local computer, and further operable to provide biofeedback or other feedback or assistance to the subject, wherein the subject is assisted in achieving a health-related goal.

### REFERENCE TO RELATED APPLICATIONS

This application is a continuation in part of U.S. Patent Application Ser. Nos. _____, filed Jan. 8, 2003, and U.S. Pat. No. 01/41689, filed Aug. 13, 2001; and also claims priority from U.S. provisional application Ser. Nos. 60/347,134, filed Jan. 9, 2002 and 60/362,030, filed Mar. 6, 2002, the content of each application being incorporated herein by reference.

### FIELD OF THE INVENTION

The invention relates to systems and methods directed to maintaining or improving the health of an individual.

### BACKGROUND OF THE INVENTION

There is high demand for systems and methods to assist persons achieve improved health. For example, obesity is a common problem in the developed world. Metabolic rate meters, such as indirect calorimeters, can be used to measure metabolic parameters of a person and assist in the development of weight control programs. Indirect calorimeters are described, for example, in U.S. Pat. Nos. 6,402,698, 6,468,222, 6,309,360, 6,277,645, 6,135,107, 5,836,300, 5,179,958, 5,178,155, 5,038,792, 4,917,108.

Physiological monitoring systems, methods, and devices, which may be used in embodiments of the present invention, are described in Int. App. Nos. WO01/89368, WO01/82789, WO01/52718, WO01/39089, WO01/28416, and published U.S. application Ser. Nos. US20,010,049,470 and US20,010,044,588.

Calorie management systems and methods, elements of which may be used in embodiments of the present invention, are described in Int. App. Nos. WO01/89365, WO01/82783, WO01/28495, and WO01/26535, U.S. application Ser. No. 09/721,382, and U.S. Pat. No. 6,478,736.

Patents and patent applications referred to in this specification are incorporated herein by reference.

### SUMMARY OF THE INVENTION

Embodiments of the present invention provide improved systems and methods to assist persons achieve health-related goals, such as weight control.

A system for assisting a person achieve a health related goal comprises a physiological monitor, providing a physiological parameter of a subject; a local computer, adapted to receive the physiological parameter from the physiological monitor; and a remote computer, in communication with the local computer over a communications network, adapted to receive the physiological parameter from the local computer, and further operable to provide biofeedback to the subject, so that the subject is assisted in achieving a relaxed state. The physiological parameter may be correlated with the metabolic rate (for example, heart rate, body temperature, skin conductivity, respiration rate, oxygen consumption, carbon dioxide production, metabolic rate determination, muscle activity, brain activity, or other physiological parameter) of the subject, and the physiological monitor may be an indirect calorimeter, a heart rate monitor or other physiological monitor. The biofeedback may comprise an audiovisual presentation, wherein the audio or visual component of the audiovisual display is correlated with the metabolic rate of the subject. The audiovisual presentation may be provided to the user using audiovisual capabilities of the local computer, or using an audiovisual device, separate from the local computer (such as a wireless phone, interactive television, other television, Internet appliance, or the like), wherein the audiovisual device has a communications link with the communications network..

The remote computer may also provide coupons to assist the person in achieving a weight loss goal, authorization to use a fitness center, or other feedback.

In this application, the term health-related goal includes health maintenance, health improvement, weight control, fitness improvement, prevention of disease, physiological goals, and the like. For example, health-related goals may include blood pressure control, blood glucose control, cardiac health improvement, stress reduction, weight loss, diet improvement, and physical fitness improvement. A health related goal may include a number of factors, such as reduction of body fat percentage combined with an increase in resting metabolic rate.

The term weight control includes weight loss, weight maintenance, and weight gain, according to a person's requirements.

The term fitness center, as used herein, refers to a location where a person can receive advice (such as fitness advice, health related advice, dietary advice, diet planning, medical advice), use equipment (such as exercise machines, medical equipment, weighing scales, body fat meters, indirect calorimeters, or other physiological monitors), measure their metabolic rate, determine other physiological parameters, or receive other services. For example, a person may exercise, receive counseling, receive nutrition advice, receive health advice, undergo tests, use equipment, and have physiological parameters determined. A fitness center may be a diet club, gym, weight control program, resort, spa, medical facility, sports or recreational facility, location provided by an organization that the person has joined to assist the achievement of health-related goals.

### BRIEF DESCRIPTION OF THE FIGURES

FIG. 1 shows a system according to an embodiment of the present invention;

FIG. 2 shows a method according to an embodiment of the present invention, where a person attends a fitness center;

FIG. 3 shows a method according to an embodiment of the present invention;

FIG. 4 shows a system according to an embodiment of the present invention for use in a corporate wellness program or similar program;

FIG. 5 shows a system according to an embodiment of the present invention for use in a nursing home or similar facility;

FIG. 6 shows a relaxation system; and

FIG. 7 shows a system for providing biofeedback over a communications network.

Elements of systems and methods from different examples may be combined or exchanged according to requirements.

Health Improvement System

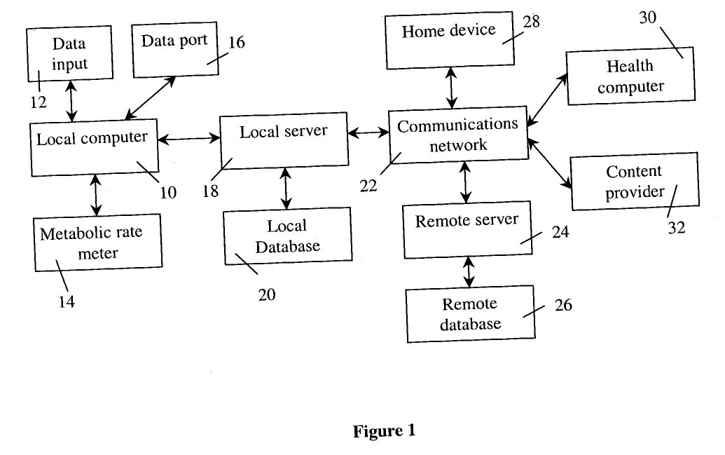

FIG. 1 shows an embodiment of a system to assist a person achieve health-related goals, comprising a local computer 10, data input device 12, metabolic rate meter such as an indirect calorimeter 14, data port 16, local server 18, local database 20, communications network 22, remote server 24, remote database 26, home entertainment device 28, health professional's computer 30, and content provider 32.

Data relating to the person can be stored in remote or local databases. Data can include the person's body weight, body fat percentage, height, age, weight, dietary information, health-related targets, fitness level, VO₂, and the like.

In one embodiment, the local computer is in communication with a metabolic rate meter. The person measures their resting metabolic rate, and the value is transmitted to the local computer and subsequently stored in a database. As described in more detail below, the local computer can be provided by a fitness center such as a gym or weight control center.

For the purposes of weight control, the person's resting metabolic rate is determined using the metabolic rate meter 14. The meter 14 is in communication with the local computer 10, for example using a cable connection, or wireless link such as Bluetooth, IEEE811(b) or other wireless protocol. Metabolic parameters, and other personal data, can be stored within a local database 20 or remote database 26.

The data input device 12, such as a keyboard or menu entry system can be used to enter demographic, physical, physiological and other data relating the person (personal data), such as height, weight, body fat percentage, age, gender, ethnic origin, goals (such as related to weight, health, and fitness), hydration, cardio-vascular health, fitness levels, the person's identity, insurance information, and the like. The data port 16 can be used to download other personal data from other devices, such as physiological monitors or activity monitors carried by the person, or data from any other device (such as a personal digital assistant, PDA) having relevant information related to the person. In other embodiments, a single computer can replace the local computer and local server of FIG. 1.

The remote server can be provided by a weight control business affiliated with the fitness center providing the local server. The remote server receives data from the local server over the communications network 22. The remote server may receive data relating to the person from other sources, for example directly from the person over the communications network, or from other sources. The remote server uses the data to generate, for example, a weight control program for the person. The elements of the weight control program can be viewed by the person, for example as a web page accessible over the communications network. The web page can be generated by the remote server so as to have icons or other visual components associated with the fitness center, such as trademarks, graphics, design, and trade dress appearance. The remote server may be provided by a health-related business that is affiliated with, but separate from, the business owning the fitness center. The remote server may use proprietary algorithms and software to generate, for example, a weight control program for the person.

Hence, the business owning the fitness center need not know the methods by which the weight control program is produced. However, the program provided to the person can be presented to the person in a manner which shows an affiliation with the fitness center business, for example through use of visual elements on a web site used to display the program.

For example, a weight control business may provide the indirect calorimeter, the remote server, and a secure connection between the remote server and a fitness center. The person measures their resting metabolic rate and other physical parameters at the fitness center. This personal data is stored on a local database, and also transmitted to the remote server of the weight control business over the communications network. The weight control business provides a weight control plan to the person, accessible to the person as a web page. The plan can be presented as a service of the fitness center, which can give the presentation of the plan a more familiar appearance to the person. Data relating to the plan can be stored locally on the fitness center computer. Plan goals, directives, nutrition advice, and the like can be transmitted from the remote server to the local server. Data relating to the person can updated at intervals, and used to modify the goals of the weight control plan, for example by synchronization of data stored at the local computer and remote computer at intervals.

In other embodiments, only one database is used to store data relating to the person, which can be a local database, for example located on a fitness center computer, or a remote database accessed through a communications network from a fitness center computer.

A home entertainment device 28, such as a home computer or interactive television, can be used by the person to provide additional or updated data (for example, the person's current weight). The person can be provided with software, run on a computing device at the person's home, that prompts the person to enter required data, which is then transmitted over the communications network 22 to one or more databases, for example located on the local server 18 or remote server 24. In other embodiments, a web page generated by either local server 18 or remote server 20 can be used to display information relating to the weight control plan, and to provide forms or similar data entry mechanisms to receive updated data from the person. The person may also provide updated information, such as weight, using a wireless phone, for example by calling a certain number, then pressing keys correlated with the required data. The received data can then be used to update plan goals, or to initiate the provision of feedback, for example audiovisual feedback viewed on an interactive television. The fitness center computers can also be used by the person to update data or receive feedback.

The appearance of a web page generated by the remote server can be changed according to the computer (as known from the IP address) used to display it. For example, a computer at the fitness center may show a web page with prominent visual elements associated with the fitness center, whereas a home computer display may show other visual elements, such as those related to the business providing the remote server, advertisers, physicians, or other providers of goods or services.

The person may also visit a health professional, such as a doctor or nutritionist, who may review the weight control program using health professional computer 30 having a connection to the communications network 30. The health professional can be given authorization to modify the weight control program, so that the special expertise of the health professional is incorporated into the weight control plan. The web page displayed to the user may have icons or other visual identifiers associated with the health professional or a related organization.

A person may also update their records using voice recognition systems. For example, an interactive television may have voice recognition capability, and generate alphanumeric data from e.g., spoken responses of the person to questions such as “What is your current weight?” A person may also call a telephone number and answer questions, the answers being processed by voice recognition software so as to update records corresponding to the person, and also to update any health-related goals.

Fitness Center

In this example, the person visits a physical location called a fitness center. The fitness center can provide access to an indirect calorimeter, which is used to determine metabolic parameters of the person, such as resting metabolic rate (RMR) and activity-related energy expenditure. A health professional at the fitness center can assist the person in obtaining an accurate resting metabolic rate value. The fitness center can provide access to a local computer system, which can form part of a health improvement or health maintenance system, for example as described above in relation to FIG. 1.

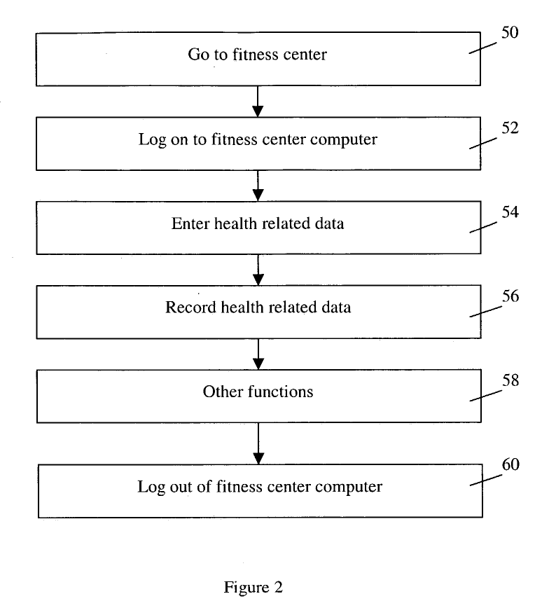

FIG. 2 shows an example of a method to assist a person achieve health-related goals. Box 50 corresponds to a person visiting a fitness center. Box 52 corresponds to the person logging into a fitness center computer. Box 54 corresponds to the person entering health-related data into the fitness center computer. Box 56 corresponds to recording health-related measurements, such as resting metabolic rate. Box 58 corresponds to other functions of the fitness center computer, which may include the provision of customized health-related feedback to the person based on data entered, activity planning, exercise program planning, viewing progress towards goals, analyzing health-related data, ordering nutritional supplements, arranging appointments with health professionals, arranging appointments with personal fitness trainers, and the like. Box 60 corresponds to logging out of the system.

To log into the system, the person may enter a password, swipe an identity card, or provide some other identification. Health related data entered at step 54 may include height, weight, age, and the like. During an initial visit, and at later intervals, a person may enter or update (as appropriate) information such as name, address, other personal information, weight control goals, and the like. During other visits, the person may only update limited data, such as those which may be expected to change during a health-related program, for example weight or metabolic rate data.

The recording of health related measurements at step 56 can include making measurements using devices in communication with the local computer, or in proximity so that data can be entered in as measured. For example, the local computer can be in communication with weighing scales, a body fat meter, and an indirect calorimeter. In response to prompts on the screen of the local computer, the person uses the devices to determine data, which is then transmitted to the local computer.

Other functions, at step 58, can further include the provision of feedback, advice, analysis of data, and questionnaires designed to establish the person's lifestyle preferences, and hence reasonable goals.

The person can view a display on the computer screen, which can be generated by the fitness center computer, by a local server, or by a remote server accessed over a communications network such as the Internet. For example, the display can be a website generated by the remote server and having characteristics (such as icons, trademarks, graphics, text, or other visual identifiers) associated with the fitness center. For example, a remote server that is associated with an affiliated weight control business can generate the display.

The person may also view the display from other devices, such as a home entertainment devices such as a computer or interactive television.

Hence, a method of providing a weight control program to a person comprises: determining data relevant to weight control of a person (such as height, weight, age, metabolic rate, activity levels, weight control goals, and the like) at a first location associated with a first business (such as a fitness center), providing the data to a first computer associated with the first business, transmitting the data over a communications network to a second computer associated with a second business (such as a remote server associated with a weight control business), analyzing the data using a software program running on the second computer, and using the second computer to generate feedback relevant to weight control (such as a diet plan, exercise plan, or weight control program in the form of a web page) viewable by the person over the communications network, wherein the information is generated by the second computer associated with the second business, and has visual identifiers associated with the first business. This method has the advantage of allowing the second business, which may, for example, have particular expertise in analyzing the results from a metabolic rate meter, to provide technical support to the first business, while retaining familiar visual identifiers of the first business in the presented results.

Home Use

The person can access the system from the person's home or other location, such as a doctor's office, dietitian's office, or from the office of another health professional.

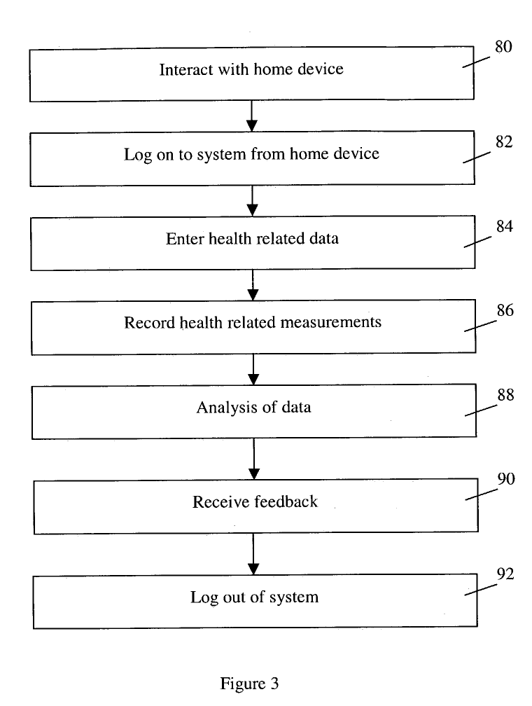

FIG. 3 shows an example of a method to assist a person to achieve a health-related goal. Box 80 corresponds to the person interacting with a home entertainment device, such as a computer, Internet appliance, interactive television, or other device. A telephone, PDA, or other electronic device may also be used.

Box 82 corresponds to the person logging on to the system, for example using a password, cookie, script program, or other appropriate method.

Box 84 corresponds to the person entering health-related data, for example as described above in relation to box 54 of FIG. 2. This may include the person's weight, body fat percentage, metabolic rate, or other physiological data. Data may be entered using voice-recognition technology.

Box 86 corresponds to entering health-related measurements, these are typically physiological parameters that can be conveniently measured at home, and may include body fat percentage. For example, as the person sits in front of the entertainment device, physical parameters can be determined by an appropriate measuring system and transmitted to the entertainment device. Devices in the home can further transmit data to the entertainment device, for example body weight scales can have a wireless transmission method of transmitting data.

Box 88 corresponds to analysis of recorded data, for example using charts, graphs, and the like on the display of the entertainment device. A remote control or personal digital assistance (PDA) can be used to control the display of data, and also to transmit numerical and other data to the entertainment device. The person can watch videos, for example transmitted from a content provider (for example, as shown at 32 in FIG. 1) through a communications network, according to the progress the person has made within a health-related program.

Box 90 corresponds to the person receiving feedback through the entertainment device; the enhanced audiovisual capabilities of an interactive television are ideal for this purpose. The privacy of home use enables the advice to be highly personalized without the risk of privacy invasion by other users of, for example, a fitness center.

Box 92 corresponds to the person logging out of the system; this may occur automatically after a certain time period after the last data entry or data viewing has been carried out.

Setting Up A Health Improvement Account

A person can set up an account using professional assistance, for example at a fitness center. Computer assistance can also be used.

For example, while at a fitness center, the person accesses a computer as a new user, and is guided through steps so as to set up a new health improvement program. This can include entering data such as the person's height, weight, age, gender, metabolic rate, body fat percentage, weight goals, body fat goals, calorie budget, or other data.

For example, for a weight control program, a person can be encouraged to undergo a measurement of their resting metabolic rate if they are not in possession of this information. The measurement of resting metabolic rate can be carried out at the fitness center, for example with the assistance of a health professional. Other measurements can be recommended, such as a metabolic rate correlations with activity levels, for example as described in more detail in my previous application. Once a weight control program has been developed, can be stored on the remote server, and accessed from home over the communication network, and printed out at the fitness center, so as to assist the person in achieving their goals. The fitness center can provide professionals, so as to assist the person set up their health improvement program.

System Using PDA

A person can carry a portable electronic device or system, such as a personal digital assistant (PDA). The PDA can be used for diet logging, for example using software such as described in U.S. Pat. Nos. 5,704,350 and 4,891,756 to Williams, and incorporated herein by reference, and my previous patents. The diet logging software can be used to record food consumption, and the data transmitted to a remote server, for example using a communications network such as the Internet. Cable or wireless methods such as data synchronization can be used to transmit data from the PDA to a home computer, other home entertainment device, fitness center computer, or other computer, so that the data can be further transmitted to a remote server, for example using a communications network. A wireless communications system can also be used to transmit data from the PDA to a remote server.

The PDA can be used to allow the person to log into a system, for example through the transmission of an identity code. The PDA can contain authorization codes to use fitness center devices such as metabolic rate meters and the like, as described in more in U.S. provisional application Ser. Nos. 60/347,134, filed Jan. 9, 2002 and 60/362,030, filed Mar. 6, 2002, the contents of which are incorporated herein by reference.

Balance Log

The person can use diet logging software, such as balance log software described in my previous applications, such as WO0182783A2 (Method and apparatus for diet control) and WO0128495A2 (Integrated calorie management system), incorporated herein by reference. A balance log database, for example of food identities and corresponding nutritional data, and personal consumption data, can be stored in a database associated with a remote server, the chip set of a set top box for an interactive television, on a PDA carried by the person, on a computer system associated with a fitness center, or elsewhere.

The person can carry a step counter, activity sensor, heart rate monitor, or other activity sensor so as to provide an activity signal correlated with activity-related energy expenditure (AEE), as discussed in more detail in my previous applications.

Calorie balance can be calculated for a person using calorie balance log software, knowing the person's metabolic rate, caloric intake, and caloric expenditure. Progress towards health goals, such as weight goals, can be monitored by the system. Feedback can be provided to the person according to their progress towards health-related goals. Rewards can also be provided, as discussed in more detail below.

Corporate Wellness Program

A system according to embodiments of the present invention can be used in an improved corporate wellness program.

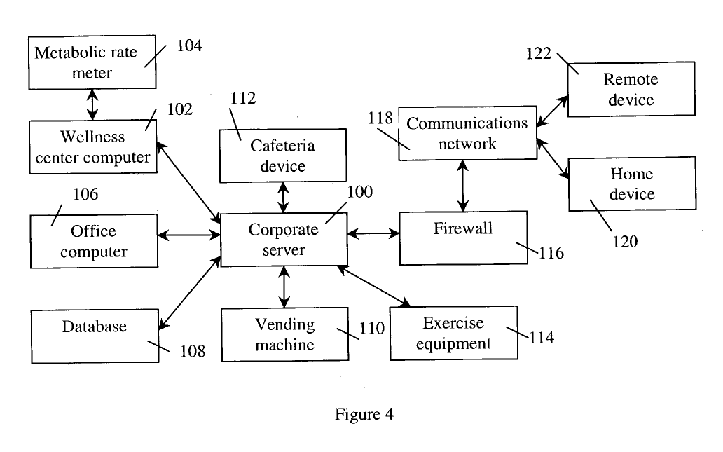

FIG. 4 shows a system comprising a corporation server 100, a wellness center computer 102, indirect calorimeter 104, office computer 106, database 108, vending machine 110, cafeteria retail device 112, exercise equipment 114, corporation firewall 116, communications network 118, employee home entertainment device 120, and remote server 122.

An employee can have a health and/or medical evaluation at the corporate wellness center, the data being entered into the computer 102 and stored in database 108. For example, the person's resting metabolic rate can be determined using a metabolic rate meter 104 (such as an indirect calorimeter), and the data transferred to the computer 102 by wired or wireless methods. The employee can access the database 108 through the office computer 106, for example using a company intranet.

Food purchases from the company cafeteria and from vending machines can be recorded, along with the person's identity, and stored in a diet log within database 108 for the person. For example, a cafeteria device 112, such as a checkout machine, can be connected to the server 100, allowing purchases by identified employees to be recorded. Similarly, a vending device 110 and exercise machine 114 can be connected to the server 110. An employee identification card can be used for purchases, allowing the identity of the purchaser to be recorded along with the identity of purchased items. The vending machine 110 and cafeteria device 112 can send data to the database 108, for example through a company intranet or other communications link. One or more exercise machines can also be connected to the server, allowing exercise data to be recorded for identified individuals.

Data can be transferred outside of the corporation firewall 116 to a remote server 122 using the communications network 118. An employee can then access data from home, for example using a home device 120, such as a computer or other entertainment device such as an interactive television. The employee may also access data from a health professional's office or fitness center using the communications network.

The employee can enter foods eaten, either using the office computer or a home computer, if the food eaten is not automatically recorded by another method. This process creates a diet log for the employee. The employee can also enter activities, and combined with metabolic rate data, the calorie balance can be determined for the employee. Nutritional analysis allows provisions of vitamin supplements to employees as needed. Activities at a company gym can be recorded automatically, and added to a health database for the employee. Exercise machines within the company gym can be connected to the company intranet, and send exercise parameters to database 108 according to the identity of the person. The person can use a company identity card to for identification by an exercise machine.

Feedback can be provided to the employee through the employee's office computer. For example, a suggested lunch menu can be e-mailed to the employee, based on food available at the cafeteria or local restaurants. Coupons for suitable foods can be provided, for use at the cafeteria, selected local restaurants, or with food delivery services.

Employees can be grouped in teams for participation in weight loss competitions. A person can view the progress of their own weight loss, together with that of team members, and that of competing teams. Peer pressure from co-workers can improve the effectiveness of a weight loss program.

Hence, an improved corporate weight control program for an employee comprises: determining a resting metabolic rate for the employee; determining a weight control goal for the employee; monitoring food purchases by the employee from corporate food retail facilities (such as a cafeteria or vending machine); monitoring activity levels of the employee (such as use of an exercise machines, or by movement if the employee is fitted with a tracking device); receiving data relating to other activities and food consumption by the employee (such as meals eaten by the employee outside of work hours); determining a calorie balance for the employee; determining the weight of the employee at intervals; and providing feedback to the employee using the employee's computer, wherein the feedback includes suggestions for meals consistent with the employee reaching the weight control goal. For example, if an employee is gaining weight, the calories consumed in high calorie snacks can be presented to the person, along with low calorie alternatives. Coupons can be provided for low calorie meals at the corporate cafeteria, or affiliated restaurants.

Nursing Home

Systems according to embodiments of the present invention can be used in nursing homes and hospitals, for patients that are not able to enter data themselves. For example, a caregiver measures the resting metabolic rate of a patient, and this value is entered or otherwise transferred to a software program on a nursing home computer. Nutritional data relating to food given to patients is also recorded. Activity levels can also be determined or estimated. A software program can then be used to calculate a calorie balance for the patient, allowing nutrition supplied to the patient to be modified as necessary to maintain, decrease, or increase the weight of a patient as required.

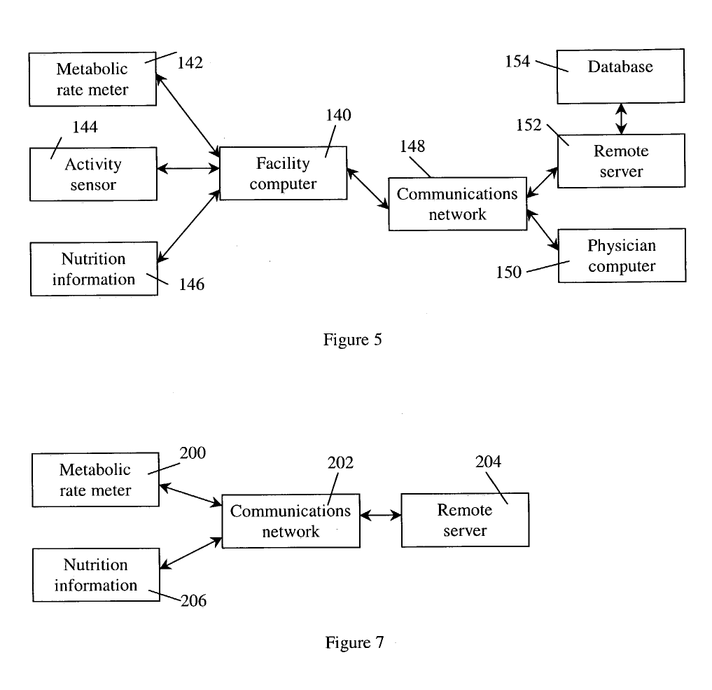

FIG. 5 shows an example of such a system, comprising a facility (such as a nursing home) computer system 140, indirect calorimeter 142, activity sensor 144, nutrition recording device 146, communications network 148, physician's computer 150, remote server 152, and database 154.

Data from the indirect calorimeter 142 is transmitted from to the computer system 140 using wireless or cable methods, along with the identity of the patient. The activity sensor 144 can be a heart rate monitor, accelerometer, or other device providing an activity signal correlated with the activity level of the patient. An activity sensor can be associated with the bed of the patient, for example a motion sensor under the patient's body, or an ultrasonic or IR based motion sensor above the patient. The nutrition-recording device 146 can be a PDA carried by the caregiver, a barcode reader (if meals provided are labeled with a bar code), or other device that provides nutritional data corresponding to meals eaten by the patient. Alternatively, meals provided to the patient can be recorded by any method, and then later entered into the computer system using, for example, a keyboard. Metabolic rate data, activity data, and nutritional data are input into a software program on the facility computer 140. The software is used to calculate the calorie balance of the patient from the difference between calories consumed as food and calories expended by resting metabolism and physical activity. The software program can also be used to monitor the nutritional balance of meals provided. The software program can also run on the remote server 152, accepting data and returning a calorie balance analysis over the communications network 148. The communications network may be the Internet. Data can also be transmitted over the communications network 148 for review by a physician, nutritionist, or other health professional using physician's computer 150. Data relating to the patient can be stored on the memory of the facility computer 140, or transmitted over the communications network for storage on the remote database 154.

Similar systems and methods can be used for hospital patients, such as intubated patients.

Coupons And Portion Size Control

Progress in a health program, such as a weight control program, or the status of a hospital or nursing home patient can be used in portion size control.

For example, a person in a weight control program can be provided with coupons for reduced portion servings at restaurants, cafeterias, and other food vending establishments and machines. A weight control business may be associated with a restaurant chain, allowing members of the weight control program to receive discounts on reduced portion servings. The portion size can be correlated with the progress that the person has made towards their weight control goals. Portion size can also be determined using the person's resting metabolic rate, or daily caloric expenditure, as more fully described in my previous applications.

Hence, an improved weight control method for a person comprises: monitoring the progress of the person towards a weight control goal; and providing coupons to the persons for meals having a calorie content related to the progress the person has made towards the weight control goal. For example, if a person's weight control goal requires a consumption of 2,000 calories per day, the person may be provided with a coupon for an 800 calorie lunch and a 1200 calorie dinner. The caloric value of the meal received using the discount coupon can be modified according to the person's daily goals, or long term progress towards a weight control goal. The meals may be smaller than standard servings, for example through the use of smaller components (such as a smaller burger), absence or substitution of components (such as omitting mayonnaise) or provision of smaller numbers of plural objects, such as a reduced number of fries.

Coupons may also be provided to a person based on their caloric needs. Hence, an improved weight control system comprises: determining the caloric needs for a person; and providing coupons to the person for meals chosen to help the person conform to the caloric needs. Caloric needs can be determined by determining the person's caloric expenditure due to resting metabolic rate, and estimating or determining the person's calorie expenditure due to physical activity. As discussed above, coupons can be provided for meals of lower caloric content than conventional servings, for example, at participating restaurants. Hence, a method of assisting a person towards a weight control goal comprises: determining the person's caloric intake requirements, and providing coupons for meals having a modified caloric content consistent with the person's caloric intake requirements.

An improved weight control method for a person comprises: monitoring the progress of the person towards a weight control goal; determining if the progress is adequate; and providing a coupon to the person for a reward if the progress is adequate. Adequate progress may include achieving a goal, substantially achieving a goal, or partially achieving a goal. The reward may be a consumable, such as an ice cream, or for reduced price. movie admission, or some other free or reduced cost goods or service.

A coupon may be a printed paper, a printable file from a computer, an authorization code (such as an electronic code), or some other method or device for allowing the person to receive goods or services on favorable terms.

Calorie balance can be calculated for a person using calorie balance log software, knowing the person's metabolic rate, caloric intake, and caloric expenditure. Progress towards health goals, such as weight goals, can be monitored by the system. Rewards can be provided to the person as goals are reached. For example, if a goal is met, a coupon can be provided to the person. Electronic coupons can be transmitted to a PDA allowing immediate use by the person on achieving a goal. For example, in a corporate wellness program, a coupon can be sent to the office computer of a person, allowing a reward to be picked up immediately, for example in a cafeteria. The coupon provided may take the form of a printed or printable document, authorization code, or some other form. Purchases can be made in person or using a communications network.

Portion size control, for example using coupons, can be used as part of health-related methods described elsewhere within this application.

Relaxation Systems

A person may visit a health center (such as a resort, spa, swimming center, sports center, medical center, fitness center, or the like) for rest, health improvement, and relaxation. During a visit to such a location, a person's resting metabolic rate can be measured while the person is in a fully relaxed state, and this value can be used to set up a customized health improvement program for the person, such as a weight control program. The measurement of RMR and the development of health improvement programs can be included in the cost of the visit, or be provided for an additional charge.

For example, in the case of weight control, the determined metabolic rate for the person in a relaxed state, in combination with estimated activity energy expenditures, allows the nutritional requirements of the person to be determined with improved accuracy. Resting metabolic rate should be determined for the person in a fully relaxed state. Relaxation methods have been discussed in a previous application to Mault. Resting metabolic rate can be determined for a person lying in a relaxed state, for example on a bed or in a fluid bath.

A relaxation system can comprise a fluid bath in which a person lies, such as a mud bath or Jacuzzi. Measurement of metabolic rate is provided to a feedback system, which is used to control one or more environmental parameters. These might include heat of the fluid, aeration of the fluid, lighting, ambient sound, vibration, aroma, or massage (by machine or by a person receiving a signal correlated with metabolic rate). For example, if the person's resting heart rate is known, a pulsing sound at that rate or lower can be provided, by which method the person's heart rate can be decreased.

FIG. 6 shows a relaxation and metabolic rate measurement system comprising a bath 160, in which a person 162 lies. The person is immersed in fluid 164, and breathes through an indirect calorimeter 166, shown having a mask 166b. The indirect calorimeter provides a metabolic rate measurement to a control system 180. The person is illuminated by lighting system 168, controlled by light controller 170. The fluid 164 is heated by heater 172, having heater control 174. The fluid is aerated by aerator 176, having aeration control 178. The person is exposed to audio signals from speaker system 180, having speaker control 182. The lighting control, heater control, speaker control, and aerator control are connected to the control unit 184, so that aeration, lighting, fluid temperature, and audio signals can be correlated with measured metabolic rate. Double-headed arrows represent either cable or wireless links between devices.

For example, as measured metabolic rate decreases, lighting may dim, the low frequency components of audio signals may increase, and aeration may reduce in gas volume per unit time. Temperature of the fluid may be held constant, or may be reduced or increased over time as metabolic rate decreases. The lighting system may be an array of light emitting diodes, and the illumination wavelength may change with measured metabolic rate.

The control unit 184 receives metabolic rate data from the indirect calorimeter, and contains a processor and memory. The time-dependence of the metabolic rate data can be analyzed, using methods disclosed in my previous applications, so as to determine if the person is close to a fully relaxed state, or when the person has been in a fully relaxed state long enough to obtain reliable data. For example, the time stability of the metabolic data can be monitored when it has reached a low level, and the measurements terminated after a certain time period, such as one or more minutes. Alternatively, the system can be used to determine which environmental parameters are most relaxing, as evidenced by a low level of metabolic rate, so that the person can use the same or similar conditions at home or elsewhere to achieve a relaxed state.

The control unit 184 provides signals correlated with metabolic rate, or the change in metabolic rate since the beginning of the test, to the other control devices. The other control devices, such as the light controller 170, change ambient conditions (in this case, lighting intensity, modulation, wavelength or spectrum, and the like) in response to the signal from control unit 184 in a predetermined way.

The control system may additionally (or alternatively) receive data relating to other physiological parameters, such as heart rate, respiration frequency, brain waves, and the like, and the ambient conditions can be changed in response to these signals.

In other embodiments, the person lies on a table while metabolic rate is measured. Biofeedback in the form of lighting, audio signals, mechanical vibration, aroma and the like can be correlated with measured metabolic rate. A video screen can be used in place of the lighting system. For example, a video of waves on water can be displayed to the person, and the wave size decreased as the person's metabolic rate falls. The size and repetition frequency of other displayed natural events can be correlated with measured metabolic rate, using conventional video processing techniques. An audio signal can contain music, natural noises, or occasional or repetitive words of encouragement, for example using voice synthesis methods.

Hypnosis can also be used to relax the person. A hypnotist can view the metabolic rate of the person, such as provided by an indirect calorimeter, and provide instructions or feedback to the person based on the determined metabolic rate value. An algorithm can be used to estimate how close the person is to a fully relaxed state, based on extrapolation of time-dependent metabolic rate data, and this information used by the hypnotist to modify the feedback provided to the person.

After determination of resting metabolic rate (RMR), the value of RMR and other physical data can be used to determine a customized health program for the person. This can be set up with the assistance of a health professional and/or a computer expert system. The person can later access the program at home, via a communications network, for example using a system similar to that described in relation to FIG. 1.

If the person fails to meet the goals established by the health program, additional visits to the health center can be scheduled for further measurements, consultation, advice, or for the determination of a revised program.

Relaxation Using Biofeedback Received Over A Communications Network

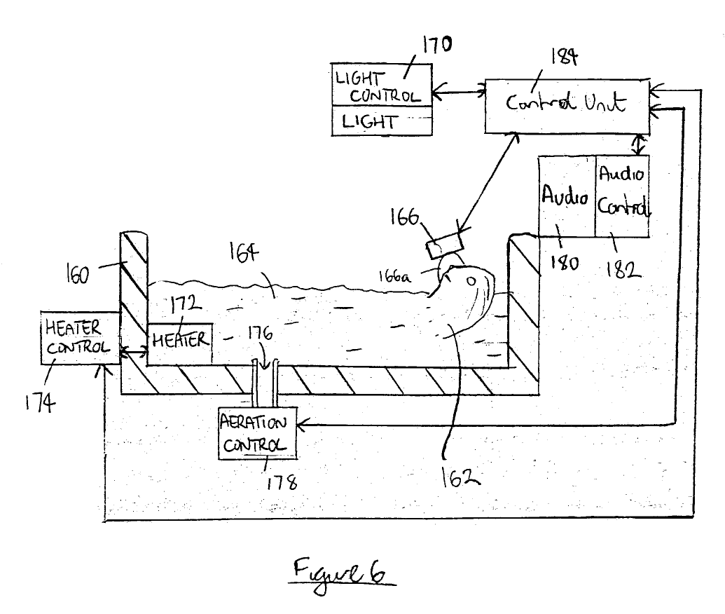

FIG. 7 shows an indirect calorimeter 200 with a connection to a communications network 202 (such as the Internet). A remote server 204 and entertainment device 206 are also connected to the communications network.

A person breathes through the indirect calorimeter, and data is transmitted to the remote server, for example through a wireless Internet connection. Data may comprise signals from the sensors in the metabolic rate meter, analyzed data corresponding to average metabolic rate over a time period, heart rate, respiration rate, body temperature, or other data. The indirect calorimeter may have a wireless connection to the communications network, or connection to another device (such as a phone, computer, or other device) having a connection to the network. Feedback, for example, audiovisual feedback, is generated by the server, and transmitted to the entertainment device for presentation to the person breathing through the indirect calorimeter. The entertainment device can be a computer, interactive television, other audio-visual device, or other electronic device. For example, audio signals and a visual presentation can be correlated with the relaxation state of the person, as determined by metabolic rate and/or other physiological parameters which may be transmitted to the server. The relaxation methods discussed in the previous section, in relation to correlation of ambient conditions and metabolic rate, can be applied to this system.

The indirect calorimeter may receive data from a heart rate sensor, and feedback to the person may be modified by the determined heart rate. If the person's heart rate increases by some value during part of the test, the metabolic rate test may be abandoned or data obtained during the period of elevated heart rate can be discarded.

Hence, an improved method of determining resting metabolic rate comprises: breathing through an indirect calorimeter; transmitting metabolic rate data to a remote server over a communications network; receiving feedback from the server over the communications network, wherein the feedback is presented on an electronic device, such as a computer, interactive television, Internet appliance, or other device. The feedback may include: video images (for example, video, animations, or computer-generated graphics having spatial and frequency components correlated with metabolic rate), audio signals (such as music, mantras, words encouraging relaxation), mechanical vibrations of devices connected to the communications network, aromas, magnetic fields, electrical fields, and the like. The feedback is correlated with the determined metabolic rate, other physiological status, or combination of physiological parameters. If the measurement is unsuccessful, a video presentation of correct indirect calorimeter use can be presented to the person.

The indirect calorimeter can contain an electronic identification of the supplier, so that the feedback, such as a web page displayed on an entertainment device, can be customized with visual elements associated with the supplier of the calorimeter to the user. A token (as described above) can be used to identify the person to the system, allowing feedback to be customized to the known physiological condition or preferences of the person).

A similar system can be used to assist the person achieve a relaxed state. For example, the person can wear a physiological monitor (such as heart rate monitor, respiration rate monitor, indirect calorimeter, EKG unit, brain wave scanner, or other physiological monitor), which provides physiological data correlated with degree of relaxation (such as heart rate data). This physiological data is transmitted over a communications network to a remote server system. The remote server system analyzes the data, and determines the degree of relaxation of the person (possibly using stored previous data for the person), and trend in degree of relaxation. Feedback is provided to the person over the communications network, for example to an entertainment device such as a computer, interactive television, and the like. For example, calming music can be played, relaxing images displayed, and encouraging spoken messages can be played.

A system such as described above can be used to prepare a person for a resting metabolic rate measurement. For example, once physiological data shows the person is relaxed, they might be told to start a metabolic rate measurement.

Described examples are not intended to be limiting. Other embodiments will be clear to those skilled in the arts.

Having described my invention, I claim:

1. A system for assisting a person achieve a health related goal, comprising:
a physiological monitor, providing a physiological parameter of a subject;
a local computer, adapted to receive the physiological parameter from the physiological monitor; and
a remote computer, in communication with the local computer over a communications network, adapted to receive the physiological parameter from the local computer, and further operable to provide biofeedback to the subject, wherein the subject is assisted in achieving a relaxed state.

2. The system of claim 1, wherein the physiological parameter is correlated with a metabolic rate of the subject.

3. The system of claim 1, wherein the physiological monitor is an indirect calorimeter.

4. The system of claim 1, wherein the physiological monitor is a heart rate monitor.

5. The system of claim 2, wherein the biofeedback comprises an audiovisual presentation, wherein the audio or visual component of the audiovisual display is correlated with the metabolic rate of the subject.

6. The system of claim 5, wherein the audiovisual presentation is provided to the user using audiovisual capabilities of the local computer.

7. The system of claim 5, wherein the audiovisual presentation is provided to the user using an audiovisual device, separate from the local computer, wherein the audiovisual device has a communications link with the communications network.
